(19) United States
(12) Patent Application Publication
(10) Pub. No.: US 2007/0275982 A1
(43) Pub. Date: Nov. 29, 2007

(54) METHOD

(75) Inventors: Meenakshi Balganesh, Bangalore (IN); Santosh Nandan, Bangalore (IN)

Correspondence Address: ASTRAZENECA R&D BOSTON, 35 GATEHOUSE DRIVE, WALTHAM, MA, 02451-1215, US

(73) Assignee: ASTRAZENECA AB, (US)

(21) Appl. No.: 11/746,821

(22) Filed: May 10, 2007

(30) Foreign Application Priority Data
May 11, 2006 (IN) 836/CHE/2006

(51) Int. Cl. A61K 31/519; A61K 31/4745; C12Q 1/18; A61K 31/17

(52) U.S. Cl. 514/259.31; 514/291; 514/594; 435/32

(57) ABSTRACT

A therapeutic agent for administration to a bacterium or to the environment thereof which agent comprises synergistically effective amounts of (i) an RNA polymerase inhibitor and (ii) an ALS enzyme inhibitor.

This application claims priority under 35 U.S.C. §119(a)-(d) to Indian Application No. 836/CHE/2006 filed on May 11, 2006.

The present invention relates to methods for the treatment of tuberculosis and to compounds and combinations of compounds for use in such methods.

Tuberculosis (Mtu) is the single largest infectious disease killer in the world that kills about 2 million people every year. Someone in the world is infected with Mtu every second and nearly 1% of the world population is newly infected with Mtu every year. Overall one third of the world's population is infected with the Mtu bacillus and 5 to 10% of people who are infected with Mtu become sick or infectious at some time during their lifetime. Drugs in use today were discovered more than 40 years ago and since then there has been no major pharmaceutical research effort to discover and develop any new therapeutic agent. There is an urgent medical need to combat this disease with drugs that will be rapidly effective against drug-resistant as well as sensitive Mtu.

Combination therapy for Mtu includes four drugs, Rifampicin, Isoniazid, Pyrazinamide and Ethambutol, given for a minimum duration of six months. Use of multiple drugs helps in preventing the appearance of drug-resistant mutants and six months of treatment helps in preventing relapse. On the other hand, multiple drug therapy and the prolonged duration of therapy are major impediments to compliance. Control programmes aimed at implementing “compliance” through DOTS (Directly Observed Therapy Short-course) exert a huge administrative burden on any treatment. At present, DOTS is available to only 25% of TB patients. Among the four anti TB drugs, rifampicin plays a major role in shortening the duration of therapy to six months and the duration increases to 18 months in case of Rifampicin resistant Mtu. See for example N. K. Jain, K. K. Chopra and Govind Prasad. Initial and acquired isoniazid and rifampicin Resistance to M. tuberculosis and its Implications for treatment Ind. L Tub., 1992, 39, 121. Also Iseman M D, MDR-TB and the developing world—a problem no longer to be ignored: the WHO announces ‘DOTS Plus’ strategy, International Journal of Tuberculosis & Lung Disease, 1998, 2, and Global Alliance for TB drug development. Scientific blueprint for tuberculosis drug development Tuberculosis 2001 81 (1):1-52.

A reduction in the duration of therapy is clearly desirable.

The present invention is based on the discovery that Rifampicin may be co-administered with an inhibitor of the Mtu acetolactate synthase (ALS) enzyme and produce synergistic therapeutic effects.

Therefore in a first aspect of the invention we provide a method of killing or controlling the growth of a bacterium which method comprises applying to the bacterium or to the environment thereof, synergistically effective amounts of (i) an RNA polymerase inhibitor and (ii) an ALS enzyme inhibitor whereby the bacterium is killed or growth controlled.

By “synergistically effective amounts” we mean that (i) and (ii) are administered in amounts that, when applied to the bacterium or to the environment thereof according to a defined treatment regime, kill or control the growth of the bacterium.

Any convenient bacterium may be used, these include mycobacteria and is conveniently M. tuberculosis, M. avium, M. intracellulare, or M. leprae, especially M. tuberculosis and drug resistant strains thereof such as multi-drug resistant Mtu and specifically rifampicin resistant Mtu

It will be appreciated that the RNA polymerase inhibitor and the ALS enzyme inhibitor are selected for their properties as inhibitors of the particular bacterium.

It will be appreciated that (i) and (ii) may be administered at the same time ie. simultaneously or at different times (consecutively) in any convenient order; provided that administration is according to a defined treatment regime.

It will be appreciated that a defined treatment regime will depend on the particular mycobacterium and will be designed to address factors such as drug resistance and in particular multiple drug resistance. Accordingly the regime may include the use of one or more additional therapeutic agents.

The defined treatment regime may conveniently comprise one or more initial phases and one or more continuation phases.

In respect of Mtu each initial phase may, by way of non-limiting example involve up to four agents such as Rifampicin (as RNA polymerase inhibitor), Isoniazid, Pyrazinamid and ALS inhibitor. Each initial phase may be of about 8 weeks duration and involve daily dosing (for example about 56 doses in total) or five times per week dosing (for example about 40 doses). Conveniently only one initial phase is used.

Each continuation phase may involve just two agents such as Rifampicin and the ALS inhibitor and be for between about 18-31 weeks duration. The total number of doses (per agent) will depend on the agents used. Conveniently only one continuation phase is used.

We set out in Reference Example 1 hereinafter drug regimens for culture positive pulmonary tuberculosis caused by drug-susceptible organisms.

Any convenient RNA polymerase inhibitor may be used. This is conveniently Rifampicin or a derivative thereof such as Rifamycin and its derivatives like Rifapentine, Rifabutine, and other inhibitors. See for example: WO-03/084965, WO-04/005298 and Lounis N & Roscigno G. “In vitro and In vivo activities of rifamycin derivatives against mycobacterial infections” in Curr. Pharm. Design, 2004,(10) 3229-3238.

Any convenient ALS inhibitor may be used. This is conveniently selected from sulphonyl ureas, imidazolinones, triazolopyrimidines, pyrimidyl-oxy-benzoates, pyrimidyl-thio-benzenes, 4,6-dimethoxypyrimidines, indole acyl sulfonamides, pyrimidyl salycylic acids and sulphonyl carboxamides. Convenient ALS inhibitors are set out for example as set out in U.S. Pat. No. 5,998,420 (Grandoni) or the references “Herbicides inhibiting branched chain amino acid biosynthesis”—Stetter, J. (ed) Springer-Verlag, Germany and references therein, and “Synthesis and Chemistry of Agrochemicals III”, 1992—edited by Don R. Baker, Joseph G. Fenyes and James J. Steffens and references therein.

Sulfonylurea compounds are particular compounds for use in the present invention.

Triazolopyrimidine compounds are particular compounds for use in the present invention.

It will be understood that the synergistic combination provided by this invention may allow the use of sub-MIC concentrations of one or both agents, which may produce the same effect similar to when either compound is used at its individual MIC. This may be a 2 to 4 fold less MIC for either or both the compounds in the combination used. In other words it may be at a concentration of up to 50% or up to 25% of the actual MIC value.

Therefore in a particular aspect of the invention the synergistically effective amounts of (i) an RNA polymerase inhibitor and (ii) an ALS enzyme inhibitor will comprise a sub-MIC concentration of one or both of (i) and (ii).

In a further aspect of the invention we provide a therapeutic agent for administration to a bacterium or to the environment thereof which agent comprises synergistically effective amounts of (i) an RNA polymerase inhibitor and (ii) an ALS enzyme enzyme inhibitor.

In a further aspect of the invention we provide a therapeutic agent as hereinbefore defined for use in the treatment of a bacterial infection in a mammal, such as a human or animal.

In a further aspect of the invention we provide a method for the treatment of a bacterial infection in a human or animal which comprises administering to the human or animal synergistically effective amounts of (i) an RNA polymerase inhibitor and (ii) an ALS enzyme inhibitor.

A particular advantage of the present invention is that it may be used to address the problem of rifampicinresistant Mtu. Rifampicin was first introduced in 1972 as an anti-tubercular drug, and is extremely effective against M. tuberculosis. Due to its high bactericidal action, Rifampicin, along with isoniazid, is the mainstay of short-course chemotherapy. Resistance to rifampicin is increasing because of widespread application and results in selection of mutants resistant to other components of short-course chemotherapy leading to MDR-TB. Singly drug resistant strains to all the agents used in short course chemotherapy has been documented in all the countries surveyed. According to WHO, HIV and TB form a lethal combination accounting for 13% of AIDS deaths worldwide.

As ALS may be essential in Gram negative bacteria, like B. mallei etc. the invention may also be used to provide broad(er) spectrum activity. Examples of Gram-negative organisms include Burkoldaria sp. such as B. mallei; Brucella sp. such as B. suis; Pseudomonas sp. such as P. aeruginosa; Neisseria sp. such as N. gonorrhoeae, N. meningitidis, etc.

Whilst we do not wish to be limited by theoretical considerations, we believe that there is an underlying biological mechanism for the observed synergism between RNA polymerase and ALS inhibitors. This may be due to enhanced levels of the cellular metabolite ppGpp, such enhancement resulting from ALS inhibition and consequent amino acid deprivation. The cellular metabolite ppGpp is reported to be a regulator of RNA polymerase activity.

Based on the above we have devised a method for the identification of novel RNA polymerase or ALS inhibitors.

Therefore in a further aspect of the invention we provide a method for the identification of an ALS inhibitor which method comprises contacting a bacterium with (i) a bacterial RNA polymerase inhibitor at a concentration less than its minimum inhibitory concentration (MIC) and (ii) a putative ALS inhibitor, determining the combined inhibitory activity of (i) and (ii) and establishing whether the test compound is an inhibitor by reference to any inhibition of the bacterium.

It will be appreciated that (i) and (ii) may be contacted with the bacterium at the same time or in any order. Conveniently the bacterium is contacted with (i) and (ii) at the same time. Any convenient bacterium may be used in the above method such as those mentioned hereinbefore. A particular strain for use in the method is Mycobacterium tuberculosis H37Rv.

The MIC of the RNA polymerase inhibitor may be established either from available data or by routine experimentation.

The concentration of the putative ALS inhibitor to be used is conveniently selected to give a meaningful indication of its activity for example when compared with the bacterial RNA polymerase inhibitor. Convenient concentrations used include those now used routinely in drug screening protocols such as about 10 μmol to 100 uM.

The identification method is useful in the pharmaceutical and agrochemical areas.

Any convenient concentration less than the MIC can be used, provided that any synergistic contribution from the test compound can be distinguished from the activity of the RNA polymerase inhibitor alone. In practice the concentration used is likely to be less than say 80% or 75% of the MIC, such as less than 60%, 50%, 40%, 30% or 20%. Less than 50% or less than 25%, such as less than 25% are particular values.

It will be appreciated that any inhibitory effect may be due to a mechanism other than ALS inhibition. Further investigation would be required to establish the actual mechanism. Such investigations could involve mechanism of action (MOA) or enzyme inhibition studies.

It will also be appreciated that any inhibitory effect may be due to the putative ALS inhibitor alone. This is conveniently monitored by performing a parallel version of the identification method but without the RNA polymerase inhibitor. In addition a parallel version of the identification method is conveniently performed without the putative ALS inhibitor. Such parallel methods act as convenient controls.

The above method may be used in an analogous manner to identify novel RNA polymerase inhibitors.

Therefore in a further aspect of the invention we provide a method for the identification of an bacterial RNA polymerase inhibitor which method comprises contacting a bacterium with (i) an ALS inhibitor at a concentration less than its minimum inhibitory concentration (MIC) and (ii) a putative bacterial RNA polymerase inhibitor, determining the inhibitory activity of (i) and (ii) and establishing whether the test compound is a bacterial RNA polymerase inhibitor by reference to any inhibition of the bacterium.

Details given above in relation to the method for identifying ALS inhibitors apply by analogy to the method for identifying RNA polymerase inhibitors.

### BRIEF DESCRIPTION OF THE DRAWINGS

FIG. 1 is a graph showing the MIC values for Isonazid (INH) and Streptomycin (Strep) when used in combination.

FIG. 2 is a graph showing the MIC values for Ethambutol (Etham) and Isoniazid (Inh) when used in combination.

FIG. 3 is a graph showing the MIC values for Rifampicin and a sulphonylurea compound (SU) when used in combination

FIG. 4 is a graph showing the MIC values for Rifampicin and a triazolopyrimidine compound (TP) when used in combination

The invention will now be illustrated by reference to the following Figures and Examples in which:

### EXAMPLE 1

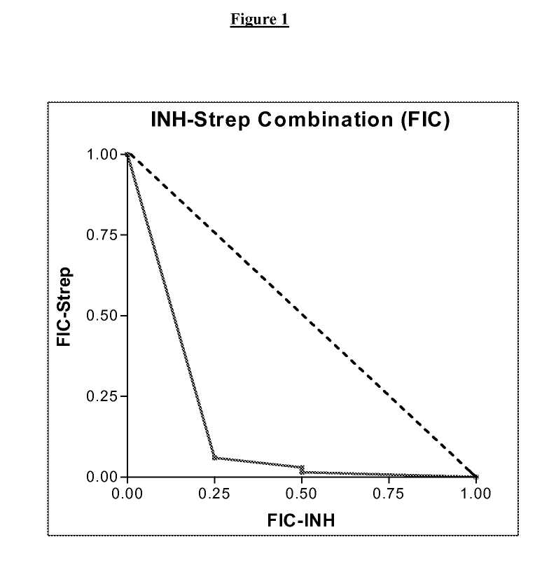

A sulfonylurea ALS inhibitor and a triazolopyrimidine ALS inhibitor were tested alone and in combination with Rifampicin. The positive controls used were Isoniazid and Streptomycin where one finds a synergistic action. The individual MICs of Isoniazid (INH) and Streptomycin (Strep) are 0.03 and 1.0 μg/ml respectively. When used in combination, these values drop to 0.0075 and 0.12 μg/ml respectively (cf. FIG. 1). This is 4 fold and 8 fold less.

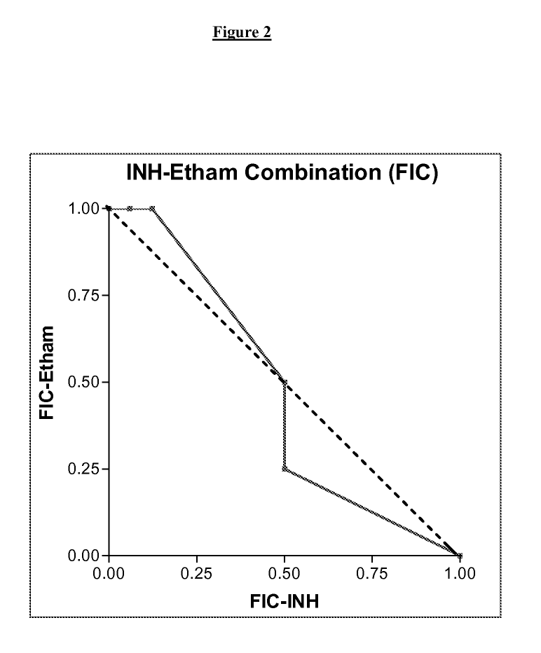

The negative control used was a combination of Ethambutol (Etham) and Isoniazid (Inh) where there is no synergistic activity. The individual MICs of 0.5 & 0.03 do not drop significantly when tested together (FIG. 2) cf. In. Clinical Microbiology Procedures Handbook; Vol. 1-2 by Isenberg, Henry. D. Ed Washington D.C.; American Society for Microbiology/1992; Pages 5.18.1 to 5.18.28).

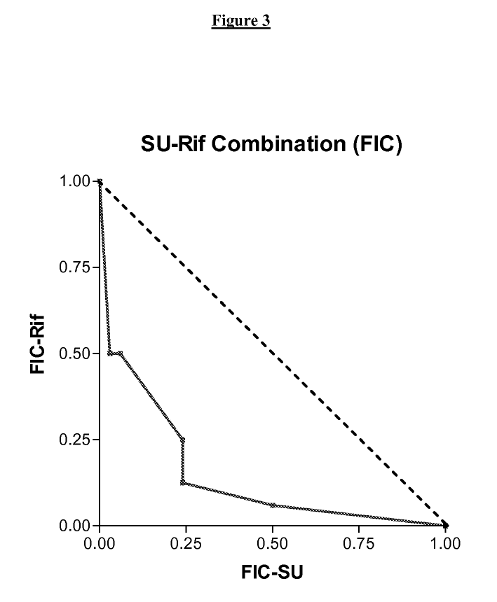

The results show clear synergy; FIG. 3 shows the individual MICs of Rifampicin and a sulphonylurea compound (SU) having ALS inhibitor activity are 0.03 and 0.25 μg/ml. When used in combination, these MICs drop 0.0038 and 0.03 ug/ml respectively, which is 8-fold less for both the drugs.

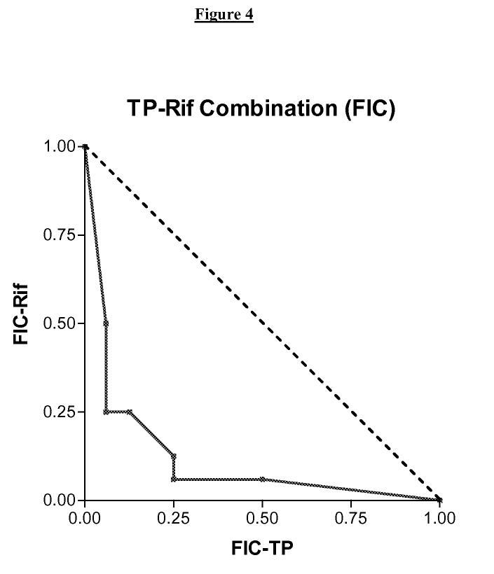

FIG. 4 shows the individual MICs of Rifampicin and a triazolopyrimidine compound (TP) having ALS inhibitor activity 0.015 & 0.5 ug/ml respectively. When used in combination, these MICs drop to 0.0038 & 0.03 ug/ml which is 4 & 8-fold less for both the drugs.

### EXAMPLE 2

### Method for the Identification of Mycobacterial RNA Polymerase or ALS Inhibitors.

The microbiology screen is performed in a microtiter plate format for screening 20-25 compounds per plate. The screen is performed using the alamar blue assay (Franzblau, S. G. et al. 1998. J. Clin. Microbiol. 36: 362-366) which provides results after 7 days.

A known ALS inhibitor is selected and used for the screen with putative RNA polymerase inhibitors. The known ALS inhibitor is used at a fixed concentration of 0.5 & or 0.25×MIC. The putative RNA polymerase inhibitors are screened at 2 concentrations, namely 10 & 100 uM. Three sets of assays are run:

1) with the ALS inhibitor alone at MIC and sub MIC concentrations which will constitute the positive control as well.

2) The unknown compounds at 10 & 100 um alone to check the inherent inhibitory activity, if any.

3) The putative RNA polmerase inhibitors at 10 & 100 um concentrations along with the ALS inhibitor at 0.5 and 0.25×MIC concentrations. Compounds which show inhibition in combination with the ALS inhibitor used at sub-MIC concentration, or enhanced inhibition when combined with ALS inhibitor, are selected for further analysis.

The same method is repeated using a known RNA polymerase inhibitor such as Rifampicin and putative ALS inhibitors.

### REFERENCE EXAMPLE 1

### Drug Regimens for Culture-Positive Pulmonary Tuberculosis Caused by Drug-Susceptible Organisms

Regimen 1 (Initial Phase)

Drugs: Isoniazid (INH); Rifampin (RIF); Pyrazinamid (PZA); Ethambutol (EMB)
Interval and doses (minimal duration): Seven days per week (wk) for 56 doses (8 wk) or 5 days/week (d/wk) for 40 doses (8 wk)
   Regimen 1a (Continuation Phase)
Drugs: INH/RIF
Interval and doses (minimal duration): Seven days per week for 126 doses (18 wk) or 5 d/wk for 90 doses (18 wk)
Ranges of total doses (minimal duration): 182-130 (26 wk)
Rating (evidence): HIV−: A (I); HIV+: A (II)
   Regimen 1b (Continuation Phase)
Drugs: INH/RIF
Interval and doses (minimal duration): Twice weekly for 36 doses (18 wk)
Ranges of total doses (minimal duration): 92-76 (26 wk)
Rating (evidence): HIV−: A (I); HIV+: A (II)
   Regimen 1c (Continuation Phase)
Drugs: INH/RPT
Interval and doses (minimal duration): Once weekly for 18 doses (18 wk)
Ranges of total doses (minimal duration): 74-58 (26 wk)
Rating (evidence): HIV−: B (I); HIV+: E (I)
   Regimen 2 (Initial Phase)
Drugs: INH, RIF, PZA, EMB
Interval and doses (minimal duration): Seven days per week for 14 doses (2 wk), then twice weekly for 12 doses (6 wk) or 5 d/wk for 10 doses (2 wk), then twice weekly for 12 doses (6 wk)
   Regimen 2a (Continuation Phase)
Drugs: INH/RIF
Interval and doses (minimal duration)) Twice weekly for 36 doses (18 wk)
Ranges of total doses (minimal duration): 62-58 (26 wk)
Rating (evidence): HIV−: A (II); HIV+: B (II)
   Regimen 2b (Continuation Phase)
Drugs: INH/RPT
Interval and doses (minimal duration): Once weekly for 18 doses (18 wk)
Ranges of total doses (minimal duration): 44-40 (26 wk)
Rating (evidence): HIV−: B (I); HIV+: E (I)
   Regimen 3 (Initial Phase)
Drugs: INH, RIF, PZA, EMB
Interval and doses (minimal duration): Three times weekly for 24 doses (8 wk)
   Regimen 3a (Continuation Phase)
Drugs: INH/RIF
Interval and doses (minimal duration): Three times weekly for 54 doses (18 wk)
Ranges of total doses (minimal duration): 78 (26 wk)
Rating (evidence): HIV−: B (I); HIV+: B (II)
   Regimen 4 (Initial Phase)
Drugs: INH, RIF, EMB
Interval and doses (minimal duration): Seven days per week for 56 doses (8 wk) or 5 d/wk for 40 doses (8 wk)
   Regimen 4a (Continuation Phase)
Drugs: INH/RIF
Interval and doses (minimal duration): Seven days per week for 217 doses (31 wk) or 5 d/wk for 155 doses (31 wk)
Ranges of total doses (minimal duration): 273-195 (39 wk)
Rating (evidence): HIV−: C (I); HIV+: C (II)
   Regimen 4b (Continuation Phase)
Drugs: INH/RIF
Interval and doses (minimal duration): Twice weekly for 62 doses (31 wk)
Ranges of total doses (minimal duration): 118-102 (39 wk)
Rating (evidence): HIV−: C (I); HIV+: C (II)

1. A method of killing or controlling the growth of a bacterium which method comprises applying to the bacterium or to the environment thereof, synergistically effective amounts of (i) an RNA polymerase inhibitor and (ii) an ALS enzyme inhibitor, whereby the bacterium is killed or growth controlled.

2. A method as claimed in claim 1 wherein the RNA polymerase inhibitor is Rifampicin or a derivative thereof.

3. A method as claimed in claim 1 wherein the inhibitor of the ALS enzyme is a sulfonylurea compound.

4. A method as claimed in claim 1 wherein the inhibitor of the ALS enzyme is a triazolopyrimidine compound.

5. A method as claimed in any one of the previous claims wherein one or both of (i) and (ii) are applied at a sub-MIC concentration for that particular agent.

6. A method as claimed in claim 5 wherein one or both of (i) and (ii) are applied at a sub-MIC concentration of no more than 50% for that particular agent.

7. A method as claimed in any one of the previous claims wherein the bacterium is a mycobacterium.

8. A method as claimed in claim 7 wherein the mycobacterium is selected from M. tuberculosis, M. avium, M. intracellulare, or M. leprae.

9. A method as claimed in claim 7 wherein the mycobacterium is M. tuberculosis or a drug resistant strain thereof.

10. A method as claimed in claim 7 wherein the mycobacterium is multi-drug resistant M. tu.

11. A method as claimed in claim 7 wherein the mycobacterium is rifampicin resistant M. tu.

12. A therapeutic agent for administration to a bacterium or to the environment thereof which agent comprises synergistically effective amounts of (i) an RNA polymerase inhibitor and (ii) an ALS enzyme inhibitor.

13. A therapeutic agent as claimed in claim 12 wherein the RNA polymerase inhibitor is Rifampicin or a derivative thereof.

14. A therapeutic agent as claimed in claim 12 wherein the bacterium ALS enzyme inhibitor is a sulfonylurea compound.

15. A therapeutic agent as claimed in claim 12 wherein the bacterium ALS enzyme inhibitor is a triazolopyrimidine compound.

16. A therapeutic agent as claimed in any one of claims 12-15 wherein one or both of (i) and (ii) are provided at a sub-MIC concentration for that particular agent.

17. A therapeutic agent as claimed in claim 16 wherein one or both of (i) and (ii) are provided at a sub-MIC concentration of no more than 50% for that particular agent.

18. A therapeutic agent as claimed in any one of claims 12-17 for use in the treatment of a bacterial infection in a human or animal.

19. A method for the treatment of a bacterial infection in a human or animal which comprises administering to the human or animal synergistically effective amounts of (i) a RNA polymerase inhibitor and (ii) an ALS enzyme inhibitor.

20. A method for the identification of an ALS inhibitor which method comprises contacting a bacterium with (i) a bacterial RNA polymerase inhibitor at a concentration less than its minimum inhibitory concentration (MIC) and (ii) a putative ALS inhibitor, determining the combined inhibitory activity of (i) and (ii) and establishing whether the test compound is an inhibitor by reference to any inhibition of the bacterium.

21. A method for the identification of an bacterial RNA polymerase inhibitor which method comprises contacting a bacterium with (i) an ALS inhibitor at a concentration less than its minimum inhibitory concentration (MIC) and (ii) a putative bacterial RNA polymerase inhibitor, determining the inhibitory activity of (i) and (ii) and establishing whether the test compound is a bacterial RNA polymerase inhibitor by reference to any inhibition of the bacterium.
